(19) United States
(12) Patent Application Publication
(10) Pub. No.: US 2010/0211023 A1
(43) Pub. Date: Aug. 19, 2010

(54) Portable drip structure

(76) Inventor: Chin-Ho Chiu, Sansia Township (TW)

Correspondence Address: Muncy, Geissler, Olds & Lowe, PLLC, 4000 Legato Road, Suite 310, FAIRFAX, VA, 22033, US

(21) Appl. No.: 12/081,111

(22) Filed: Apr. 10, 2008

(30) Foreign Application Priority Data
Apr. 16, 2007 (TW) 096206087

(51) Int. Cl. A61M 5/00; A45F 5/00

(52) U.S. Cl. 604/251; 224/148.4

(57) ABSTRACT

A portable drip structure comprises an inflating structure and a main bag. The inflating structure includes a motor and an air container and the motor and the air container are communicative with each other via a pipe provided there between, so that the air container can be inflated by the inflating structure. The main bag is provided with a first and a second compartment that neighbor with each other and each compartment is provided on the main bag with an opening that can be enclosed. The first compartment is for disposing a drip bag while the second compartment is for disposing the inflating structure. Consequently, when the two openings are enclosed and the air container is inflated via the motor, the air container will be expanded to press the drip bag in the first compartment to drip the infusion liquid in the drip bag outward.

### BACKGROUND OF THE INVENTION

### 1. Field of the Invention

The present invention relates to a drip structure and, more particularly, to a portable drip structure which is simple and easy to be operated, where the drip bag can be carried along with a patient and the infusion liquid in the drip bag can be dripped out by pressing.

### 2. Description of the Prior Art

Generally, patients are usually on a drip by using drip bags filled with different kinds of solution, such as glucose solution or other nutritional liquid, in order to help patients to recover from diseases. Besides, patients may also be on a drip by infusing slowly medication's liquid into their bodies within a period of time. For example, patients may be infused with the medical liquid of mixtures of medications and glucose solutions or saline solutions intravenously by using a drip. Therefore, drip bags are common found on drip stands in medical institutes.

In order to make patients able to displace, go to toilets, or take a walk conveniently, various portable drip stands are designed. By using such movable drip stands, patients can be easy to go around while carry drip bags and by moving the drip stands to keep the medical infusion continuously. The liquid medicine is dripped downward from the drip bags which are usually hanged on the top of the drip stands. However, such drip stands are voluminous and heavy, so that they are inconvenient to be stored and will cause inconvenience to patients who get light diseases and are able to walk around.

In order to solve the problems mentioned above, inventor had the motive to study and develop the present invention after hard research to provide a portable drip structure.

### SUMMARY OF THE INVENTION

An object of the present invention is to provide a portable drip structure that is structurally simple, easy to be operated, and convenient for users to carry.

In order to achieve the above object, the present invention provides a portable drip structure comprises an inflating structure and a main bag. The inflating structure includes a motor and an air container and the motor and the air container are communicative with each other via a pipe provided there between, so that the air container can be inflated by the motor. The main bag is provided with a first and a second compartment that neighbor with each other and each compartment is provided on the main bag with an opening that can be enclosed. The first compartment is for disposing a drip bag while the second compartment is for disposing an inflating structure. Consequently, when the two openings are enclosed and the air container is inflated via the motor, the air container will be expanded to press the drip bag in the first compartment to drip the infusion liquid in the drip bag outward.

The following detailed description, given by way of examples or embodiments, will best be understood in conjunction with the accompanying drawings.

### BRIEF DESCRIPTION OF THE DRAWINGS

FIG. 1 is an exploded perspective view of an embodiment of the present invention.

FIG. 2 is a perspective view of the embodiment of the present invention.

FIG. 2A is a sectional view of FIG. 2 along a-a′ line.

FIG. 3 is a block diagram showing the inter-relationship among components of the inflating structure of the embodiment of the present invention.

FIG. 4 is a using state diagram showing that the present invention is fastened to the waist of a user.

### DETAILED DESCRIPTION OF THE PREFERRED EMBODIMENTS

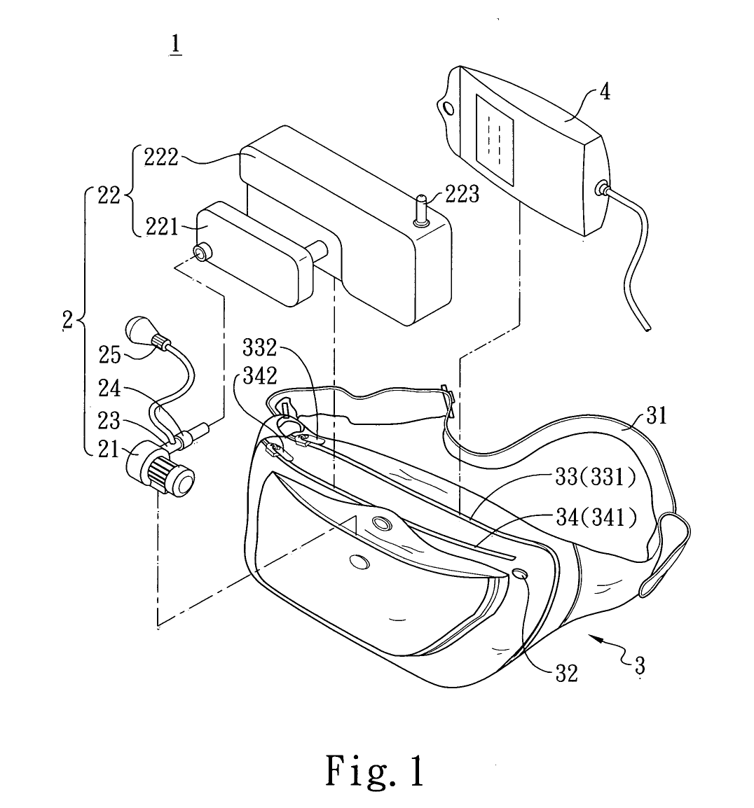

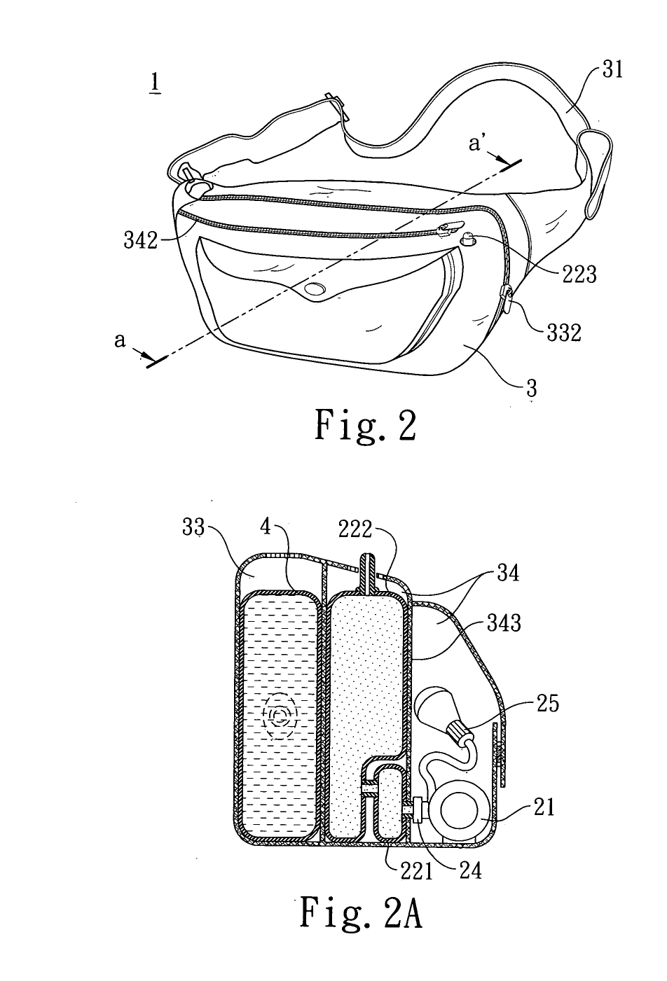

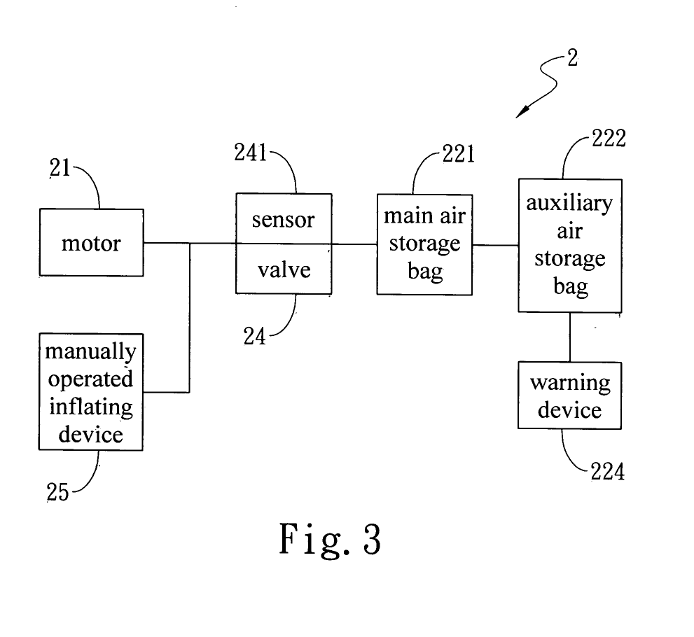

Please refer to FIGS. 1-3, which show an embodiment of a portable drip structure 1 according to the present invention. The portable drip structure 1 comprises an inflating structure 2 and a main bag 3.

The inflating structure 2 including a motor 21 and an air container 22. The motor 21 and the air container 22 are communicative with each other via a pipe 23 provided there between, so that the air container 22 can be inflated by the motor 21. The air container 22 includes a main air storage bag 221 and an auxiliary air storage bag 222. The main air storage bag 221 is in connection with the motor 21 while the auxiliary air storage bag 222 is in connection with the main air storage bag and is provided with an air outlet tube 223. A valve 24 is provided between the main air storage bag 221 and the motor 21 and is disposed with a sensor 241 for sensing the air volume in the main air storage bag 221. The sensor 241 is connected with the motor 21, so that when the air volume in the main air storage bag 221 is detected to be insufficient by the sensor 241, the valve 24 will be opened and the main air storage bag 221 will be inflated by the motor 21.

The main bag 3 includes a strap 31 for a patient to carry. Besides, the main bag 3 is provided with a hole 32 for protruding the air outlet tube 223. The main bag 3 has a first and a second compartment 33, 34 that neighbor with each other. Each compartment is provided on the main bag with an opening 331, 341 that can be enclosed. The two openings 331, 341 are disposed with zippers 332, 342 or Velcro tapes for enclosing the openings 331, 341. The first compartment 33 is for placing a drip bag 4 while the second compartment 34 is for placing the inflating structure 2. The second compartment 34 is provided with a division plate 343 therein for separating the motor 21 of the inflating structure 2 and the main air storage bag 221. Consequently, the air container 22 can be expanded to press the drip bag 4 in the first compartment 33 to drip outward the infusion liquid in the drip bag 4 when the two openings 331, 341 are enclosed and the air container 22 is inflated via the motor 21.

Therefore, as shown in FIGS. 1-3, when in practice, place the drip bag 4 into the first compartment 33, enclose the opening 331 of the first compartment 33, and inflate the main air storage bag 221 fully. When a patient is on a drip, the auxiliary air storage bag 222 is gradually inflated to press the drip bag 4 to drip outward the liquid medicine therein continuously. With the outward drip of the liquid medicine, the drip bag 4 will become flat gradually. The flat drip bag 4 can still be pressed by the auxiliary air storage bag 222 to drip outward the liquid medicine.

When the sensor 241 finds that the air volume in the main air storage bag 221 is insufficient, it will open the valve 24 and drive the motor 21 to inflate the main air storage bag 221. The valve 24 and the motor 21 will be shut off if the air volume in the main air storage bag 221 is increased to a certain amount.

Moreover, the auxiliary air storage bag 222 is connected with a warning device 224 for alerting a user that the liquid in the drip bag 4 is going to be dripped outward completely when the air volume in the auxiliary air storage bag 222 is increased to a predetermined value. The inflating structure 2 further includes a manually operated inflating device 25 in connection with the air container 22, so that a user can proceed with inflation when the motor 21 fails to work.

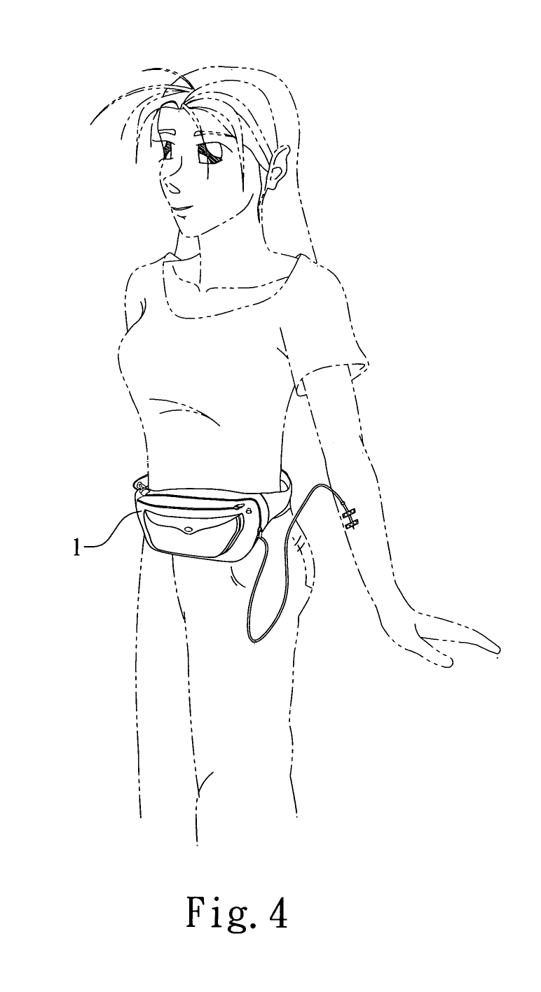

Accordingly, as shown in FIG. 4, users can conveniently carry the drip bags 4 fastened on their backs or waists via straps 31 to walk around freely without using any drip stand. Therefore, the portable drip structure can be easily carried with patients and conveniently stored and has pleasing outlook.

As disclosed in the above description and attached drawings, the present invention can provide a portable drip structure that is structurally simple and easy to operate. It is new and can be put into industrial use.

Although the embodiments of the present invention have been described in detail, many modifications and variations may be made by those skilled in the art from the teachings disclosed hereinabove. Therefore, it should be understood that any modification and variation equivalent to the spirit of the present invention be regarded to fall into the scope defined by the appended claims.

What is claimed is:

1. A portable drip structure comprising:
an inflating structure including a motor and an air container, where the motor and the air container are communicative with each other via a pipe provided there between, so that the air container can be inflated by the motor; and
a main bag carried on the body of a patient and provided with a first and a second compartment that neighbor with each other, where each compartment is provided on the main bag with an opening that can be enclosed, the first compartment is for disposing a drip bag while the second compartment is for disposing the inflating structure, and consequently the air container will be expanded to press the drip bag in the first compartment to drip the infusion liquid in the drip bag outward when the two openings are enclosed and the air container is inflated via the motor

2. The portable drip structure as claimed in claim 1, wherein the main bag is provided a strap for being carried by patient.

3. The portable drip structure as claimed in claim 1, wherein the each opening is provided with a zipper for enclosing the opening.

4. The portable drip structure as claimed in claim 1, wherein the each opening is provided with a Velcro tape for enclosing the opening.

5. The portable drip structure as claimed in claim 1, wherein the air container includes a main air storage bag and an auxiliary air storage bag, and the main air storage bag is in connection with the motor while the auxiliary air storage bag is in connection with the main air storage bag.

6. The portable drip structure as claimed in claim 5, wherein the auxiliary air storage bag is provided with an air outlet tube while the main bag is provided with a hole for protruding the air outlet tube.

7. The portable drip structure as claimed in claim 5, wherein a valve is provided between the main air storage bag and the motor and is disposed with a sensor for sensing the air volume in the main air storage bag and the sensor is connected with the motor, so that the valve can be opened and the main air storage bag can be inflated by the motor when the air volume in the main air storage bag is found insufficient by the sensor.

8. The portable drip structure as claimed in claim 5, wherein the auxiliary air storage bag is connected with a warning device for alerting user that the liquid in the drip bag is going to be dripped outward completely when the air volume in the auxiliary air storage bag is increased to a predetermined value.

9. The portable drip structure as claimed in claim 1, wherein the second compartment is provided with a division plate therein for separating the motor and the main air storage bag.

10. The portable drip structure as claimed in claim 1, wherein the inflating structure further includes a manually operated inflating device in connection with the air container.
